(19) United States
(12) Patent Application Publication
(10) Pub. No.: US 2024/0189087 A1
(43) Pub. Date: Jun. 13, 2024

(54) LOW PROFILE EMBOLIC PROTECTION DEVICE AND SYSTEM

(71) Applicant: Transaortic Medical, Inc., Morgan Hill, CA (US)

(72) Inventor: Michael T. Carley, San Jose, CA (US)

(21) Appl. No.: 18/497,510

(22) Filed: Oct. 30, 2023

Related U.S. Application Data
(60) Provisional application No. 63/432,250, filed on Dec. 13, 2022.

(51) Int. Cl. A61F 2/01; A61B 17/00; A61F 2/24

(52) U.S. Cl. CPC A61F 2/013; A61B 17/00234; A61F 2/0105; A61F 2/011; A61F 2/2427; A61B 2017/00358

(57) ABSTRACT

An example embolic protection system and method of deploying an embolic protection device are provided. An example embolic protection system for deploying a device in a diseased vessel of a vasculature comprises an embolic protection device including a catheter shaft and an expandable filter provided at a distal end of the catheter shaft. The expandable filter includes a side port provided in a side wall of the expandable filter and a filter actuator located at or towards a proximal end of the embolic protection device. The filter actuator is operable to open and close a mouth of the expandable filter. A snare guide wire is also provided in some examples.

### CLAIM OF PRIORITY

This patent application claims the benefit of priority to U.S. Provisional Application Ser. No. 63/432,250, filed Dec. 13, 2022, which is incorporated by reference herein in its entirety.

### FIELD OF THE INVENTION

The present application relates generally to devices for medical interventions conducted through vessels such as the major arteries or veins, and more particularly to devices with deployment configurations for conducting percutaneous procedures such as percutaneous valve replacement or other vascular or cardiac interventions.

### BACKGROUND

Treatment of native heart valves for conditions such as valvar regurgitation using percutaneous transcatheter procedures may involve advancing a catheter or device through the vasculature to the target native valve. The target native valve may be calcified or have other disease such as unwanted plaque or thrombus attached to the native leaflets, annulus, or other anatomical regions adjacent the native valve. Rubbing, scraping or contact between the treatment catheter and the calcifications, plaque, or thrombus can result in undesirable separation of these materials from the tissue with subsequent embolization into other parts of the body. Embolization can result in serious complications including but not limited to ischemia, stroke, tissue damage, reduced lung function, etc.

Additionally, the vessels around the heart itself, such as the aorta, may also be diseased and have similar unwanted buildups of plaque, thrombus, calcium, etc. and advancing the catheter through the vessel can also result in unwanted separation of these materials from the vessel walls with embolization. The risk of separation is exacerbated when using conventional catheters, devices, or other high-profile conventional instruments.

It would therefore be desirable to either prevent separation of the plaques, thrombus, calcium deposits, etc. from the native heart and adjacent vessels, and in situations where this does occur, capture or prevent the materials from embolizing in the patient.

### BRIEF DESCRIPTION OF THE DRAWINGS

FIG. 1 shows various portions of a diseased aorta.

FIG. 2 shows a native aortic valve AV which illustrates some of the challenges of treating a cardiac valve.

FIG. 3 shows a schematic view of a low-profile embolic protection device, according to an example embodiment. FIG. 3 includes inset views FIGS. 3A-3C.

FIG. 4 shows a schematic view of the embolic protection device of FIG. 3 used in conjunction with a dilator, according to an example embodiment.

FIG. 5 shows a schematic view of the embolic protection device and dilator of FIG. 4 used in conjunction with a loading tool, according to an example embodiment.

FIGS. 6A-6B show pictorial views of a filter dilator, according to an example embodiment.

FIGS. 7A-7B show sectional views of the filter dilator of FIGS. 6A-6B.

FIG. 8 shows a pictorial view of an atraumatic distal tip of a filter dilator, according to an example embodiment.

FIGS. 9-10 show pictorial views of open and closed configurations of a filter, according to an example embodiment.

FIGS. 11A-11D show pictorial and sectional views of a loading tool, according to an example embodiment.

FIGS. 12A-12B show respective side and top pictorial views of an example expandable introducer.

FIG. 12C shows a pictorial view of a sheath dilator, according to an example embodiment.

FIGS. 13A-13B show respective pictorial views of loaded and deployed configurations of a sheath dilator and mesh sheath of an expandable introducer, according to example embodiments.

FIGS. 14A-14B show respective sectional and enlarged part-sectional views of an expandable introducer, according to an example embodiment.

FIG. 15 shows a pictorial view of an introducer hub adapter, according to an example embodiment.

FIGS. 16A-16M show various aspects and operations in the preparation and deployment of an embolic protection device and other embolic system components for use in a medical procedure, according to example embodiments.

FIG. 17 shows a pictorial view of a therapeutic device deployed by an embolic protection device and other components of an embolic protection system, according to example embodiments.

FIG. 18 shows a schematic view of a snare guide wire used in conjunction with an embolic protection device and other embolic system components and TAVR devices in a medical procedure, according to example embodiments.

FIGS. 19A-19H show various aspects and operations in the preparation and deployment of a snare guide wire used in conjunction with an embolic protection device and other embolic system components, guide wires, and therapeutic devices (such as a TAVR device) in a medical procedure, according to example embodiments.

### DETAILED DESCRIPTION

The present disclosure describes use of the devices and methods disclosed herein during treatment in or adjacent the aorta. One of skill in the art will appreciate that this is not intended to be limiting and the devices and methods disclosed herein may be used in other anatomic regions of the body. Additionally, the present disclosure describes an embolic protection system which includes several components (e.g., a low-profile embolic protection device, an introducer, an introducer dilator, a loading tool, a dilator for the embolic protection device, and so forth). In some examples, the embolic protection system can be deployed to install a therapeutic device such as a replacement prosthetic valve in a human heart. Other uses and applications are possible in a variety of human subjects. These components may be used all together as a kit, or they may be provided individually and used individually, or they may be provided and used in any combination.

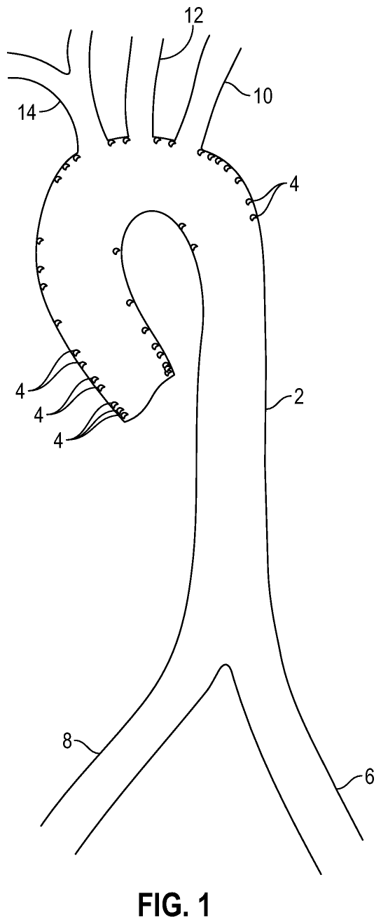

Referring to FIG. 1, an illustrative representation of a diseased aorta (2) is shown with deposits (4) distributed in several locations, including adjacent or within the left (6) and right (8) iliac arteries, and adjacent the junctions of the aortic arch with the left subclavian (10), left common carotid (12), and innominate artery (14). Navigating a diseased aorta (2) such as that depicted is a challenge with conventional intravascular diagnostic and/or interventional hardware.

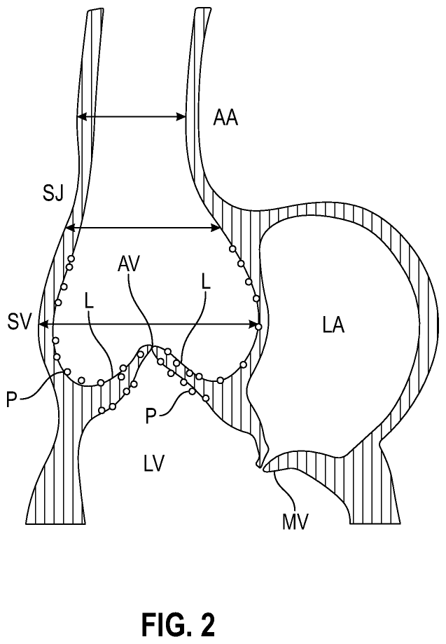

FIG. 2 shows a native aortic valve AV which illustrates some of the challenges of treating a cardiac valve. Here, the aortic valve AV includes native valve leaflets L which normally oppose one another to close the valve during contraction of the left ventricle LV to force blood away from the heart via the ascending aorta AA. The aorta adjacent the aortic valve includes the sinus of Valsalva SV and the sinotubular junction SJ. Blood is directed from the left atrium LA via the mitral valve (MV) into the left ventricle LV. The native leaflets and walls of the aorta may be covered or lined with discrete or diffuse plaques P of material such as calcium, lipids, thrombus, or other unwanted materials. When a diagnostic or therapeutic device is advanced through the vasculature toward the aortic valve, the device may scrape or otherwise contact the plaques P and cause the plaques to separate from the tissue and then embolize downstream into other parts of the body and cause complications including ischemia, stroke, etc. It would therefore be desirable to provide methods and devices that prevent the unwanted separation of plaques from the native valve, aorta, or other adjacent tissue that could embolize, and in the event that embolization does occur, it would be desirable if such methods and devices capture the emboli and prevent them from flowing downstream. While this example emphasizes the aortic valve, one of skill in the art will appreciate that this is not limiting and the devices and methods disclosed herein may be used in other heart valves such as the mitral or tricuspid valve, or they may be used in other valves of the body including venous valves, or other organs and anatomical structures in the body.

In some examples, an embolic protection system for deploying a device in a diseased vessel of a vasculature is provided. An example embolic protection system comprises an embolic protection device including: a catheter shaft; an expandable filter provided at a distal end of the catheter shaft, the expandable filter including a side port provided in a side wall of the expandable filter; and a filter actuator located at or towards a proximal end of the embolic protection device, the filter actuator operable to open and close a mouth of the expandable filter.

In some examples, the embolic protection system further comprises a dilator.

In some examples, the embolic protection system further comprises a loading tool.

In some examples, the embolic protection system further comprises an introducer.

In some examples, the embolic protection system further comprises an introducer hub adapter.

In some examples, the introducer is an expandable introducer including a mesh sheath of expandable porous mesh material; an introducer hub including a hemostasis valve; and a sheath dilator for dilating the mesh sheath, the sheath dilator being insertable into the introducer hub and including, at a distal end of the sheath dilator, a sheath dilator tip which, in a loaded configuration of the sheath dilator covers a distal open end of the mesh sheath.

In some examples, a method of deploying an embolic protection device into a vasculature of a patient is provided. An example method comprises establishing access to the vasculature; introducing a guide wire into the vasculature to assist with guidance of the embolic protection device through the vasculature; advancing the embolic protection device into the vasculature and over the guide wire toward a target treatment region, wherein the embolic protection device comprises a catheter shaft, an expandable filter disposed at a distal end of the catheter shaft wherein the expandable filter includes a side port provided in a side wall of the expandable filter, a filter actuator disposed adjacent a proximal end of the catheter shaft, wherein the filter actuator is operable to open and close a mouth of the expandable filter; actuating the filter actuator thereby radially expanding the expandable filter at the target treatment region and opening the mouth of the expandable filter; and capturing emboli in the expandable filter.

In some examples, the method further comprises inserting a therapeutic device through the embolic protection device and advancing the therapeutic device toward the target treatment region.

In some examples, the target treatment region comprises a native aortic valve.

In some examples, the method further comprises, after inserting the therapeutic device through the embolic protection device, closing the expandable filter, and withdrawing the embolic protection device from the vasculature.

Referring to the accompanying drawings, various aspects of deployment steps and configurations utilizing examples of the present embolic protection devices and systems are now described.

In some examples, the method further comprises introducing a snare guide wire into the vasculature to assist with guidance of a guide wire or a device through the side port of the embolic protection device.

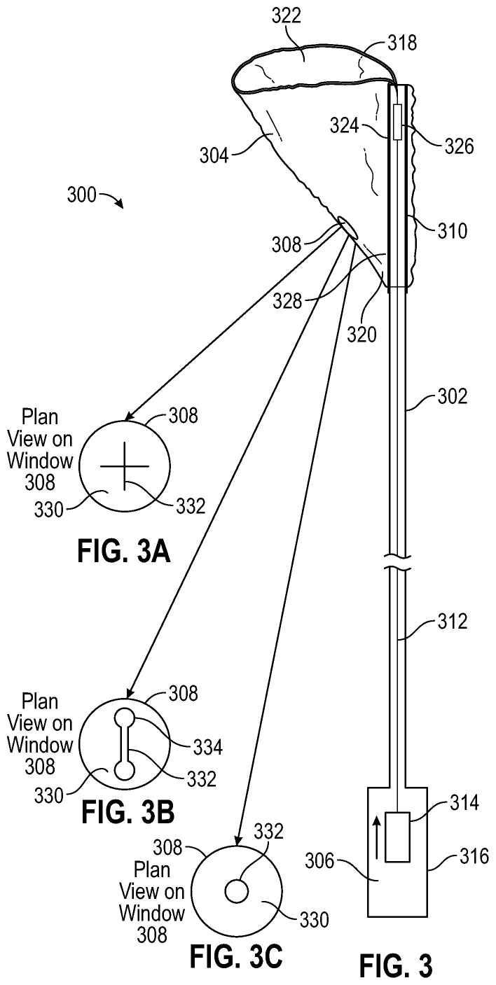

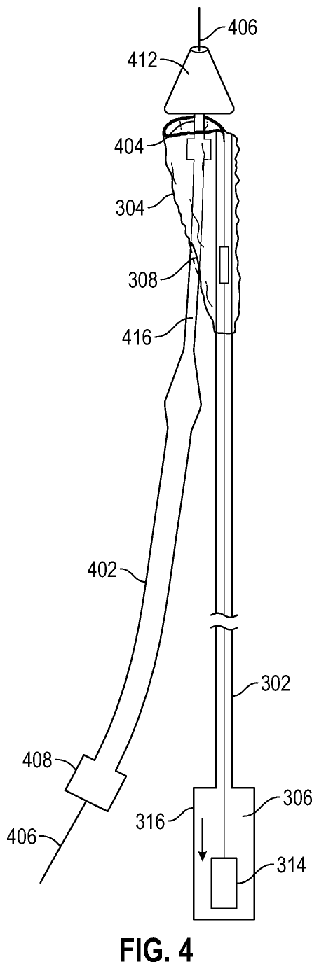

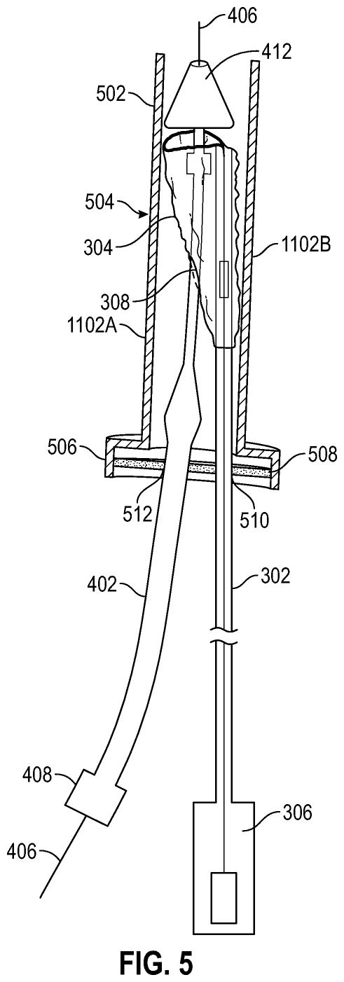

Examples of an embolic protection system may include, with reference to FIG. 3, an embolic protection device 300 that includes a catheter shaft 302, a filter 304 provided at a distal end of the catheter shaft 302, and a filter actuator 306 located at or towards a proximal end of the embolic protection device 300. With reference to FIG. 4, further examples of an embolic protection system may include a filter dilator 402 insertable through a side port 308 provided in the filter 304 of the embolic protection device 300. With reference to FIG. 5, further examples of an embolic protection system may include a loading tool 502. In some examples, the loading tool 502 comprises a peel-away loading sleeve 504 mountable around an outside of the embolic protection device 300 to assist in loading the embolic protection device 300 into a hub of an introducer, for example, as described further below. In some examples, each of these components may be provided or used separately, or provided and used in combination, for example as part a kit.

Reference is now made to FIG. 3 of the accompanying drawings which shows a schematic view of an example “low-profile” embolic protection device 300, according to an example of the present disclosure. In this application, the low-profile embolic protection device 300 is also referred to simply as an “embolic protection device 300”. In some examples, such as in the illustrated example, the embolic protection device 300 is provided as a component of an embolic protection system.

As shown further in FIG. 3, the embolic protection device 300 comprises a flexible spine 310 mounted to a distal end of the catheter shaft 302. The flexible spine 310 is hollow and supports the filter 304 in use. The flexible spine 310 carries an actuation wire 312 that extends through the catheter shaft 302 from a proximal location at an actuation slider 314 provided in a handle 316 of the filter actuator 306 to a distal location where it is connected to a hoop wire 318 of the filter 304. In some examples, the flexible spine 310 includes a stainless steel braided polymer material.

In some examples, the catheter shaft 302 is very slender (needle-like) and is highly flexible, having an outer diameter in the range 0.020-0.040 inches, and an inner diameter in the range 0.007-0.018 inches. The small outer dimensions of the catheter shaft 302 contribute to the “low-profile” characteristic of the embolic protection device 300. In some examples, a usable length of the catheter shaft 302 between the filter actuator 306 and the proximal end of the filter 304 is in the range 60-100 cm but this length may be shorter or longer, as required to suit different sizes of human anatomy. A suitable material for the catheter shaft 302 may include nitinol, stainless steel, polyethylene, polypropylene, a fluorinated polymer, polyurethane, or a stainless steel braided polymer, for example.

As shown in FIG. 3, the filter 304 is provided at the distal end of the embolic protection device 300. In this view, the filter 304 is shown in an open or expanded configuration. The filter 304 is opened and closed by manipulation of the actuation slider 314 in the handle 316 of the filter actuator 306, as described more fully below. In some examples, the filter 304 is provided as a closable basket or net. The filter 304 includes braided filter material, or a mesh or net filter material to capture (when open) and retain (when closed) embolic and other waste materials, such as plaque flakes and blood clots. The filter 304 may have a porosity that permits blood or other fluids to pass through the filter while retaining the embolic or other materials. Polyester, polyurethane, nylon, nitinol, or other materials may be used for the filter material. The filter may be coated with heparin, hydrophilic polymer, silicone, or another material to prevent platelet adhesion and/or increase lubricity.

In some examples, the filter 304 includes a frustoconical shape when flat, for example when open substantially as shown in FIG. 3. Other shapes and configurations of the filter 304 are possible. In some examples, with reference to FIGS. 4-5 for example, a shape or configuration of the filter 304 allows it to assume an elongate tubular or narrow snake-like form when collapsed, for example when a mouth 322 of the filter 304 defined by the hoop wire 318 at the distal end of the filter 304 is closed while the filter 304 is being repositioned or withdrawn from a site, for example.

In some examples, a proximal end 320 of the filter 304 is permanently closed and is fixed to a proximal location of the flexible spine 310, as shown in FIG. 3. Other fixture locations are possible. The closed proximal end 320 of the filter 304 allows capture and retention of embolic material while the filter 304 is being repositioned or withdrawn from a site, for example.

As mentioned above, the mouth 322 of the filter 304 can be opened and closed by an operator manipulating the actuation slider 314 of the filter actuator 306. When manipulated, movement of the actuation slider 314 serves to apply tension to or relieve tension from the actuation wire 312 to which it is connected. Corresponding movement of the actuation wire 312 in turn opens and closes the mouth 322 of the filter 304, as desired.

In the illustrated example, the filter 304 is supported in the embolic protection device 300 by the flexible spine 310. The flexible spine 310 may be comprised by or include an elongate thin tube 324 having an outer diameter in the range 0.050-0.100 inches and an inner diameter in the range 0.030-0.050 inches. The narrow outer dimensions of the flexible spine 310 also contribute to the “low profile” characteristic of the embolic protection device 300. The flexible spine 310 is mounted to the distal end of the catheter shaft 302, for example as shown in FIG. 3.

The flexible spine 310 carries the actuation wire 312. At its distal end, the actuation wire 312 is connected at 326 to the hoop wire 318. When the actuation slider 314 is pushed forward (or distally) towards the location of the filter 304, the hoop wire 318 expands and opens, under action and control of the actuation wire 312, the mouth 322 of the filter 304, as shown. In this position, the filter 304 is open. The mouth 322 of the filter 304 is resizable and closable by manual operation of the actuation slider 314, as needed. Conversely, retracting the actuation slider 314 pulls the actuation wire 312 in a proximal direction which applies a tension to the hoop wire 318 to collapse and close, if desired, the mouth 322 of the filter 304. Here, the filter 304 is fully closed to retain captured embolic material safely within its volume while the embolic protection device is relocated or withdrawn for example.

In some examples, the hoop wire 318 is formed integrally as an extension of the actuation wire 312. In some examples, the hoop wire 318 is formed as a separate hoop, for example as shown in FIG. 3. The mouth 322 of the filter 304 may include, or be attached to, self-expanding members to bias the mouth 322 to an open configuration. In some examples, the mouth 322 of the filter 304 may include, or be attached to, self-contracting members to bias the mouth 322 to a closed configuration. In some examples, the mouth 322 of the filter 304 can be closed and held in a circumferential groove or channel 404 provided at or towards the distal end of the filter dilator 402, for example as shown in FIG. 4. In this entrapped, closed position of the filter 304, the embolic protection device 300 can be deployed, in a “low-profile” manner, to or from the site of an operation in a diseased vessel. At the site of the operation, the filter 304 can be opened to capture embolic material that may be separated from the vessel walls. In some examples, the self-expanding or self-contracting members may cooperate to entrap the mouth 322 in, or release the mouth 322 from, the circumferential channel 404.

As mentioned above, the filter 304 includes a side port 308. In some examples, the side port 308 is formed in a wall of the filter 304, or formed as an aperture in the filter material. The side port 308 is located at or towards the proximal end 320 of the filter 304. This proximal location leaves a trapped interior volume 328 of the filter 304 that is devoid of a port or openable mouth to form a “dead zone” that can safely retain separated embolic material substantially risk free from release from the filter 304 during movement or deployment of the embolic protection device 300.

With reference to FIG. 4, in some examples, the side port 308 allows therethrough passage of a guide wire 406, and/or the filter dilator 402 (for example, disposed on the guide wire 406 as shown), and/or the passage of other devices to allow such devices (or other tools and instruments) to travel through the side port 308 and move into, or pass completely through, the filter 304 as needed for deployment at the site of an operation.

In some examples, the side port 308 includes a seal 330. The seal 330 operates to assist in retaining embolic material within the filter 304 notwithstanding allowing passage through the filter 304 of the filter dilator 402, or another device. The inset views FIGS. 3A-3C of FIG. 3 show three examples of a seal 330 provided in the side port 308. An example seal 330 may include a silicon material, for example a silicon disc as shown, or other flexible sealing material. In some examples, the silicon disc of the seal 330 may include one or more deformable holes, apertures, or slits 332 through which the filter dilator 402 or other device may pass in sealing manner. A cruciform or cross-shaped arrangement of one or more slits 332 is shown in inset view FIG. 3A. A slit 332 having a dog bone shape is shown in inset view FIG. 3B. The slit 332 of dog bone shape includes stress-relieving formations 334. FIG. 3C shows a hole 332. Other side port configurations may include a flap or other sealing member that opens and closes to allow passage of devices. Other arrangements and configurations of the side port 308, the seal 330, the one or more slits 332, and the stress-relieving formations 334 are possible.

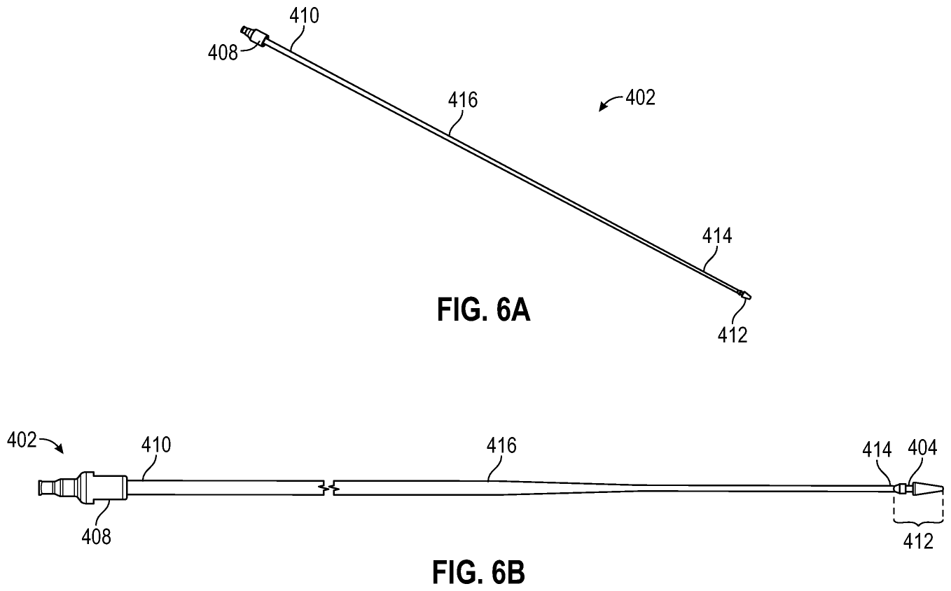

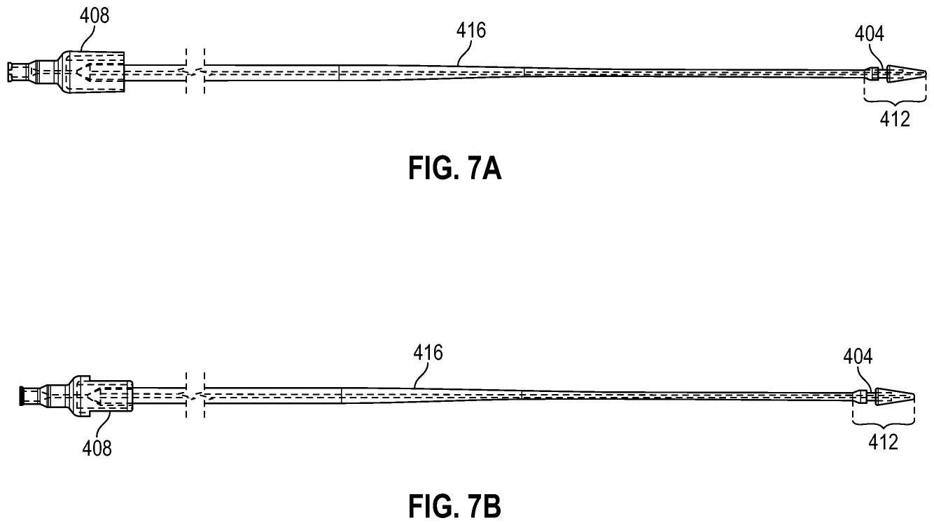

As mentioned above, FIG. 4 also includes a schematic view of a filter dilator 402. FIGS. 6A-6B show further pictorial views of an example filter dilator 402, with some of parts of the filter dilator 402 shown in enlarged view in FIG. 6B. Sectional views of the filter dilator 402 are provided in FIGS. 7A-7B.

In FIGS. 6A-6B, the example filter dilator 402 includes a Luer fitting 408 provided at a proximal end 410 of the dilator, and a filter dilator tip 412 provided at a distal end 414 of the filter dilator 402. The filter dilator 402 includes a flexible shaft 416 comprised of one or more polymers (e.g., polyethylene, polypropylene, ABS, PVC, FEP, combinations thereof, etc.).

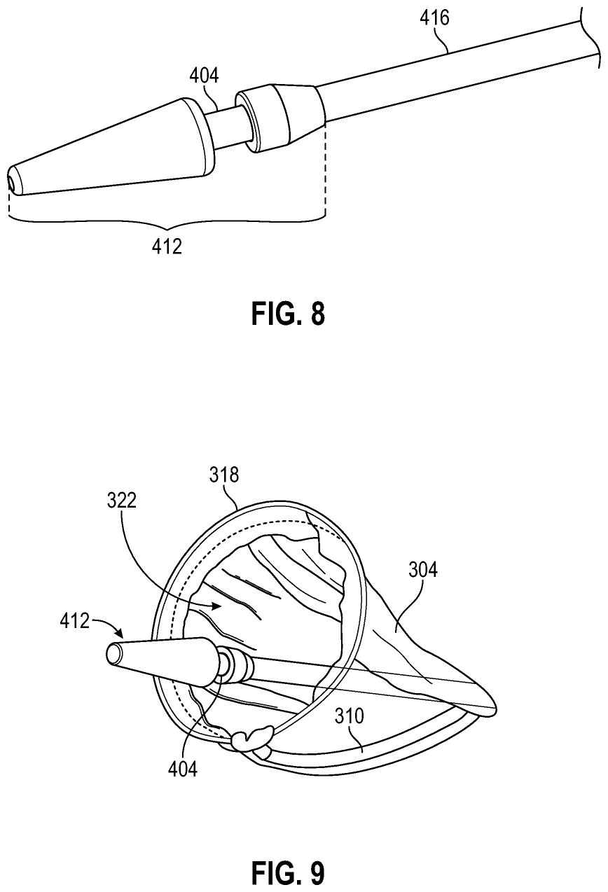

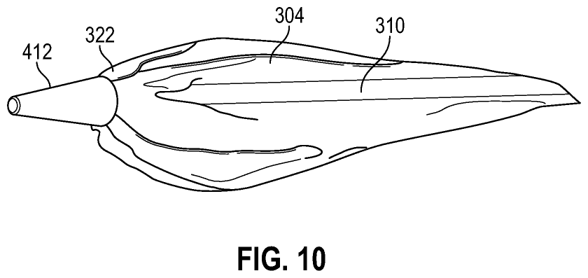

With reference to FIG. 8, the filter dilator tip 412 of the filter dilator 402 is coupled to the flexible shaft 416 and as mentioned above includes a circumferential channel 404 (or other formation) that can engage with and retain the hoop wire 318 and the mouth 322 of the filter 304 when the filter 304 is closed. Pictorial views of open and closed configurations of the filter 304 are shown in FIGS. 9-10, respectively.

In FIG. 10, under a pulling action of the actuation slider 314 away from the filter 304, the mouth 322 of the filter 304 can be captured when closed and retained within the circumferential channel 404 of the filter dilator tip 412 under locking tension in the actuation wire 312 and the hoop wire 318, respectively. The locking tension can be maintained by engaging a slider locking mechanism (not shown) of the filter actuator 306. The closed mouth 322 of the filter 304 can be securely held in the circumferential channel 404 of the filter dilator 402 thereby to support the filter 304 axially along its length to prevent collapse when the filter 304 is advanced into the human vasculature and through the confines of an introducer or loading tool.

With reference again to FIG. 5, the embolic protection device 300 may be used in conjunction with a loading tool 502. In some examples, the loading tool 502 may be referred to as a peel-away loader in view of its ability to be removed from the embolic protection device 300 after use. The loading tool 502 has three main purposes.

First, the loading tool 502 enables the embolic protection device 300 to be inserted into a hub of an introducer, as is described more fully below. An introducer can be used to insert the embolic protection device 300 (and other devices and instrumentation) into the human vasculature, typically over a guide wire 406. The loading tool 502 facilitates loading of the embolic protection device 300 into an introducer. In some examples of the present disclosure, an embolic protection system includes an adapter for a hub of the introducer to facilitate interaction and use of the loading tool 502 with various types of introducer which, in some examples, may be conventional or generic. The field of use and applications of the embolic protection devices and systems described are expanded, accordingly. An example introducer and adapter hub are described further below.

A second purpose of the loading tool 502 is to enable flushing of the embolic protection device 300 and the filter dilator 402 before introduction into the human vasculature. This allows the components to be de-aired and to prevent air bubbles from entering a patient's bloodstream. The loading tool 502 may be fitted with a flushing port, accordingly (not shown for simplicity of illustration in FIG. 5). A third purpose of the loading tool 502 is to prevent blood loss during insertion of the embolic protection device 300 and the filter dilator 402 into the introducer and/or the human vasculature.

In some examples, the loading tool 502 includes a removable hub 506. In some examples, the removable hub 506 may be peeled away from the loading tool 502. The hub 506 includes a removable seal 508 to prevent blood loss. In some examples, the removable seal 508 includes two apertures 510 and 512 allowing passage of the catheter shaft 302 of the embolic protection device 300 and the filter dilator 402 through the hub 506 while the apertures 510 and 512 remain in sealing engagement with the outer walls of these two components.

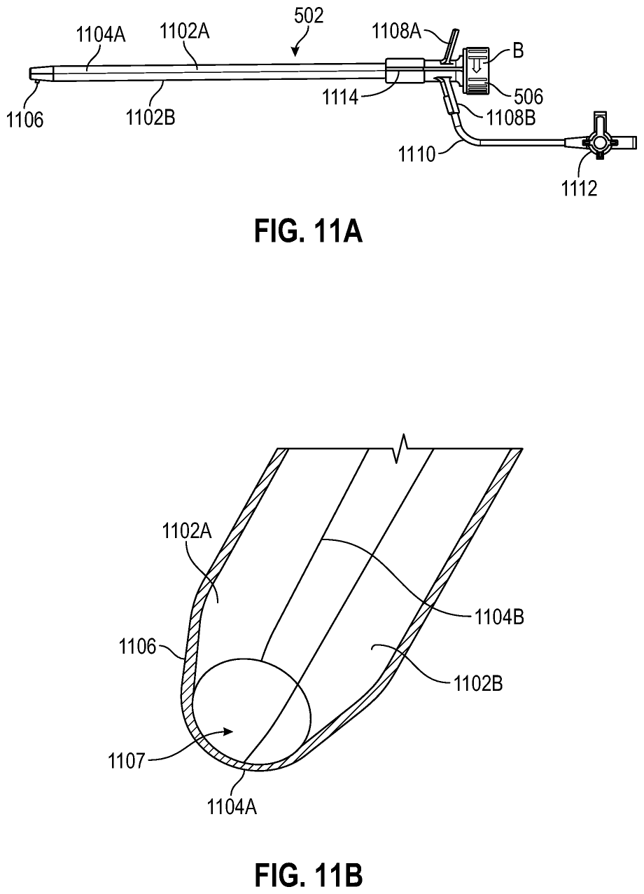

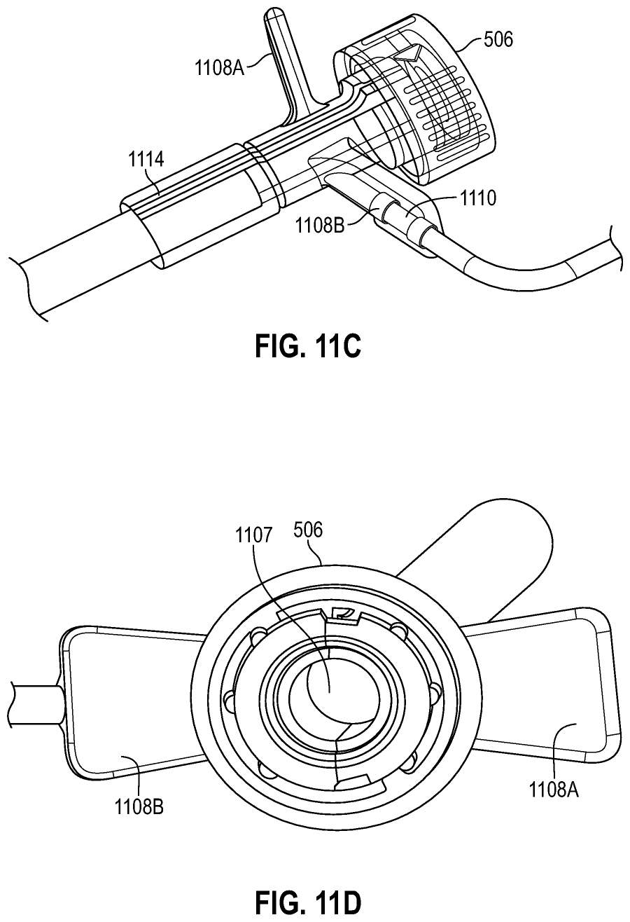

Reference is now made to FIGS. 11A-11D of the accompanying drawings which include pictorial and sectional views of another example of a loading tool 502 for the embolic protection device 300. The loading tool 502 is of tubular shape defined by two separable (or peel away) parts 1102A and 1102B, each semi-circular in cross section and joined along frangible parting lines 1104A and 1104B (seen best in FIG. 11B). The joined parts 1102A and 1102B together define a tip 1106 and a lumen 1107 of the loading tool 502 into which the embolic protection device 300 can be inserted and readied for use. The separable parts 1102A and 1102B can be split apart and peeled away from the embolic protection device 300 by manipulating two separation handles 1108A and 1108B. In the illustrated example of FIG. 11C, the handle 1108B includes a flushing line 1110 in fluid communication with the three-way stopcock 1112 and the lumen 1107 of the loading tool 502.

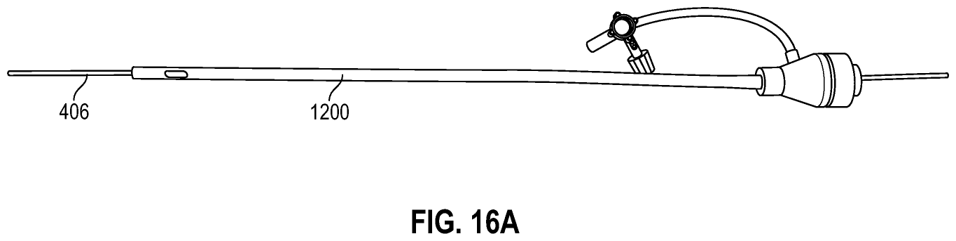

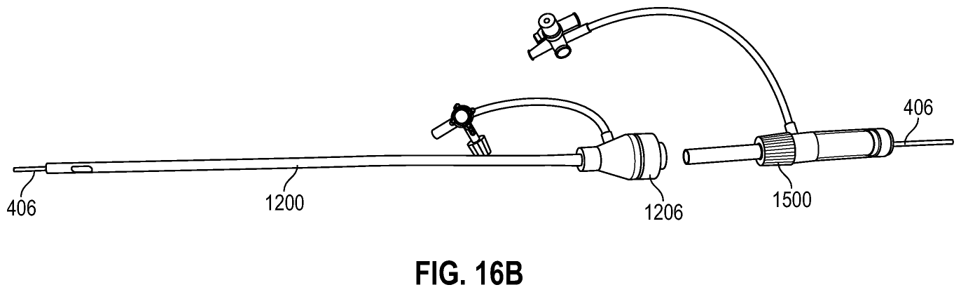

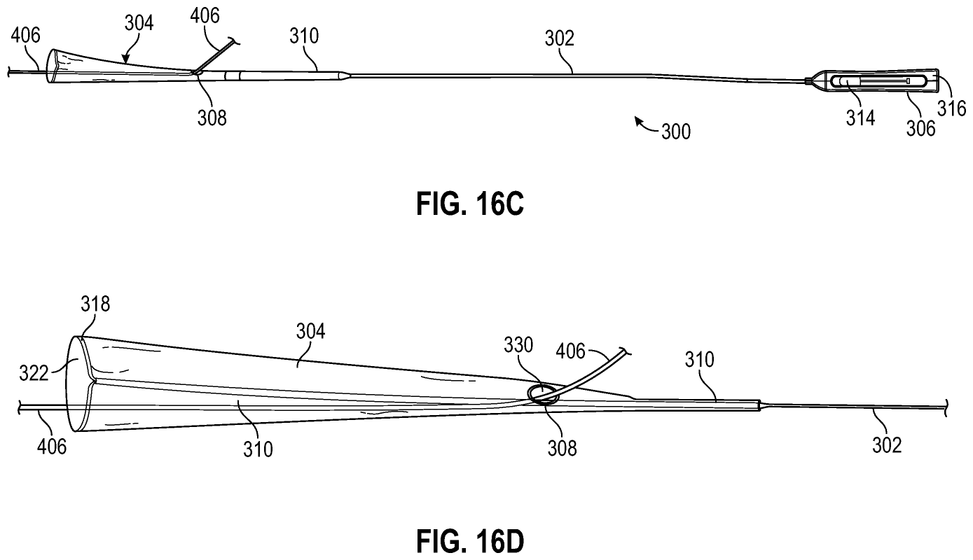

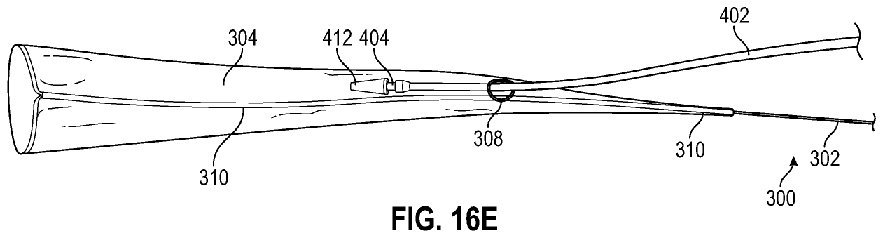

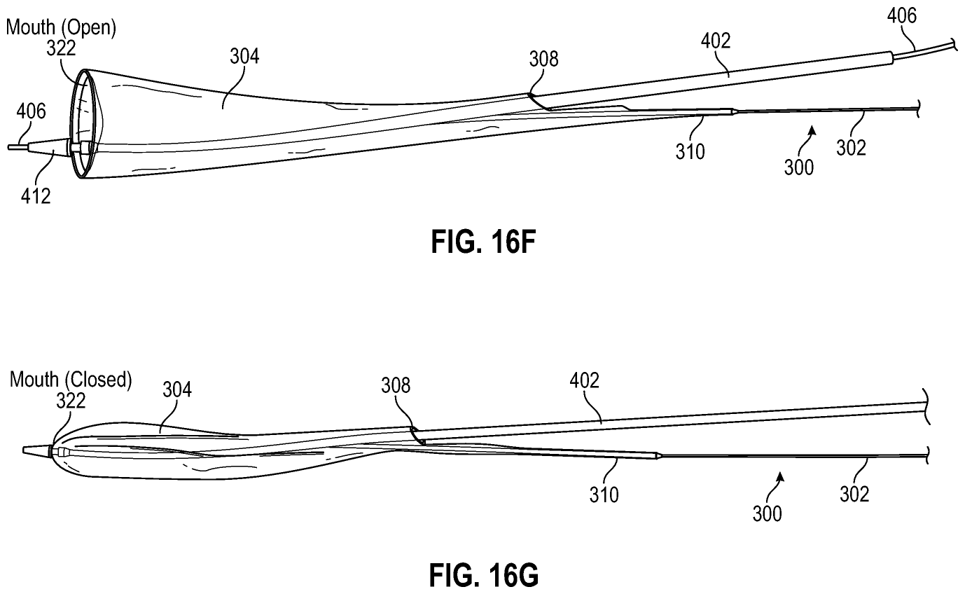

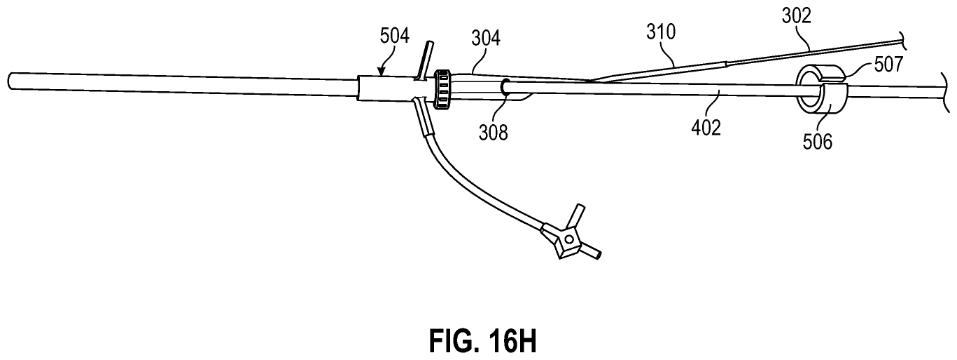

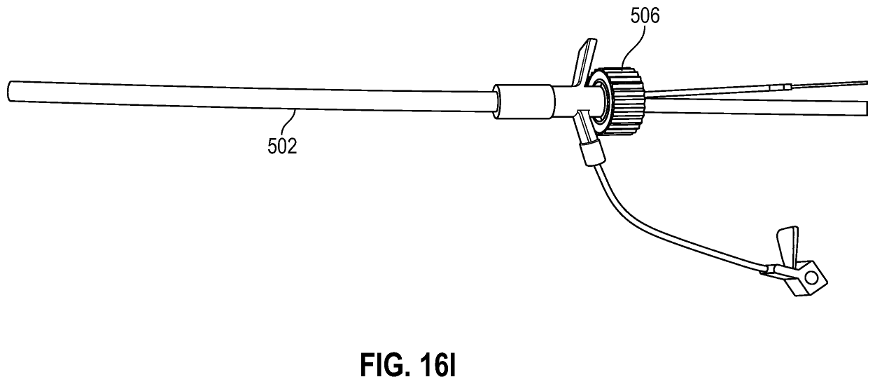

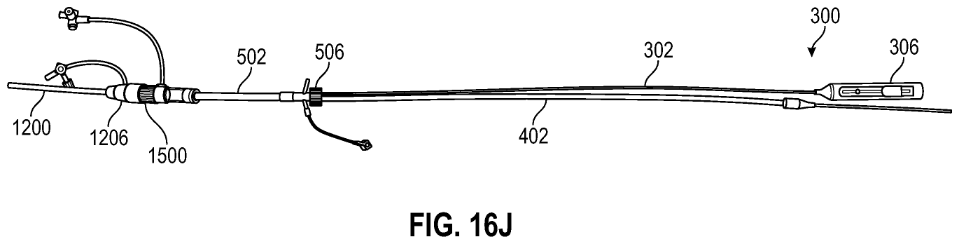

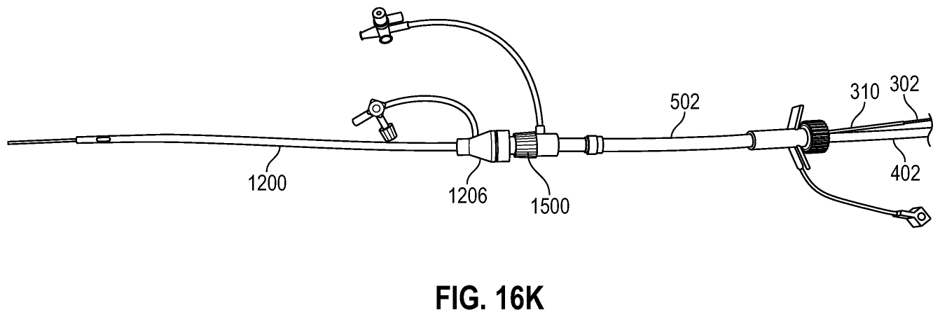

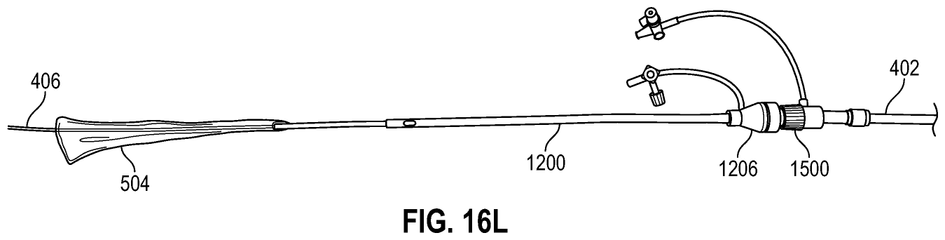

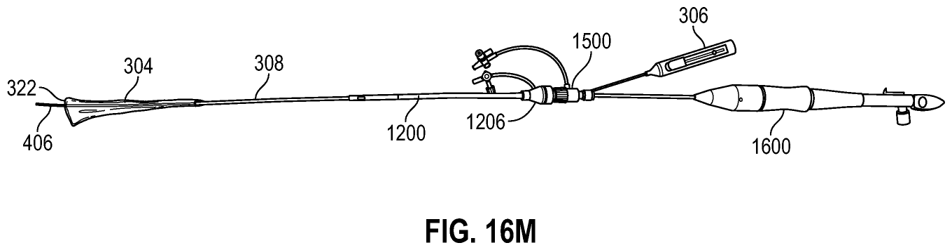

As shown, the separation handles 1108A and 1108B are joined to each other by two parting lines 1114 which are coincident with the parting lines 1104A and 1104B. Movement of the separation handles 1108A and 1108B to initiate removal of the loading tool 502 can be unlocked by rotating the hub 506 that retains (when locked) the two halves together and prevents inadvertent separation. Removal of the hub 506 from the loading tool 502 allows the separation handles 1108A and 1108B to be twisted by an operator and split apart along the parting lines (which may be perforated or have a frangible parting line), thus allowing subsequent splitting apart and removal of the parts 1102A and 1102B to release the loading sleeve completely from the embolic protection device 300. In some examples, the hub 506 can simply be eased off the separable parts 1102A and 1102B to allow their separation (peel away). In some examples, the hub 506 includes a slit or gap (507 in FIG. 16H) in its peripheral wall allowing the hub 506 to be removed from the guide wire 406 after release from the loading tool 502.

As mentioned above, the embolic protection device 300 may in some examples be inserted into the human vasculature using an introducer. The introducer may be conventional or generic. An adapter for an introducer hub may be provided. In some examples, the introducer is an expandable introducer.

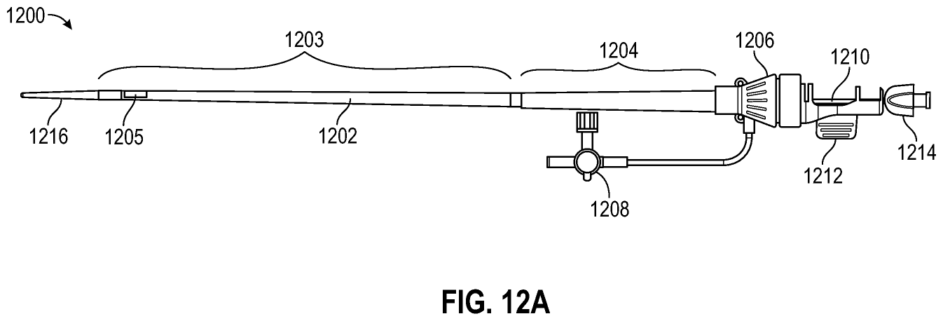

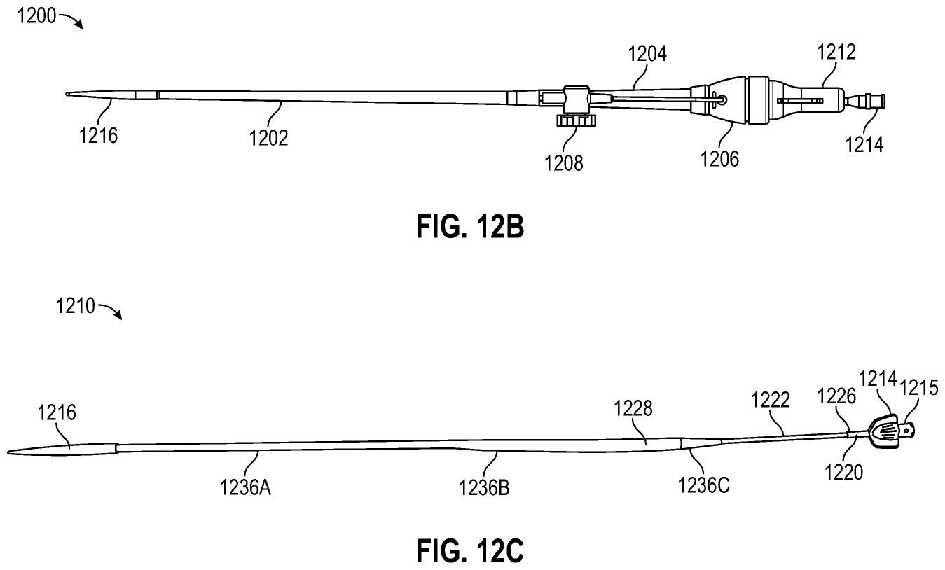

FIGS. 12A-12B show respective side and top pictorial views of an example expandable introducer 1200. The expandable introducer 1200 includes a mesh sheath (or sleeve) 1202 of expandable, porous mesh material. In some examples, the mesh material is expandable in the radial and longitudinal directions of the mesh sheath 1202. In some examples, the mesh material is expandable only in the radial direction of the mesh sheath 1202. In some examples, the mesh material is expandable only in the longitudinal direction of the mesh sheath. In some examples, the mesh sheath 1202 has a usable length of approximately 30 cm to suit an arterial dimension of the human vasculature. Some examples of this disclosure include a mesh sheath having a usable length in the range 25-35 cm to accommodate variations in vasculature size. In some examples, the mesh sheath 1202 has a contracted diameter in the range 3-7 mm and an expanded diameter in the range 7-10 mm.

In some examples, a first region 1203 of the mesh sheath 1202 is expandable and porous. The first porous region 1203 may include mesh material that is expandable in the radial direction only, and may or may not be expandable axially. In some examples, the material properties of the mesh sheath may be selected so that there is no axial expansion or contraction. In other examples, the material properties may be selected so that there is some axial expansion or contraction. In some examples, as the mesh radially expands, the mesh may foreshorten 10 mm or less in the axial direction. Other arrangements are possible. In some examples, the mesh material of the first porous region 1203 includes open pores through which fluids may pass (such as blood) while embolic material such as plaque and blood clots are prevented from passing through the mesh sheath 1202. In other examples, the mesh may not be utilized to capture embolic material but will still allow blood to pass through the membrane so that blood flow is not disrupted. A suitable mesh material for the first porous region 1203 may include polyester, Nylon, or Nitinol mesh. Pore sizes may be provided in the range 70-300 microns to allow blood to pass through the pores while capturing emboli or other particulates. At a distal end of the first porous region 1203 of the mesh sheath 1202, a marker 1205 may be provided. The marker 1205 may be radiopaque, echogenic, or visible under other imaging techniques known in the art. The marker 1205 may facilitate positioning of the expandable introducer 400 in use.

In some examples, a second region 1204 of the mesh sheath 1202 is non-expandable and non-porous. The second region 1204 may include a non-porous elastomer seal material. The second non-porous region 1204 may include a continuation of the mesh material of the first porous region 1203, but the presence of the elastomer seal material renders the second region 1204 non-porous and it may be expandable or non-expandable. In an example, the second region 1204 may be expandable but less than the first region where the mesh is disposed. The sealed second non-porous region 1204 of the mesh sheath 1202 does not allow the passage of fluid or embolic material through the walls of the mesh sheath 1202. In some examples, the second non-porous region 1204 of the mesh sheath 1202 has a length of approximately 11 cm. Other lengths are possible to suit different applications and sizes of human vasculature.

Relative to the first porous region 1203, the second non-porous region 1204 of the mesh sheath 1202 may be held in or assume an expanded or partly expanded configuration of the mesh material, as shown. The first porous region 1203 and the second non-porous region 1204 of the mesh sheath 1202 may taper down in a distal direction along their lengths as shown to facilitate advancement of the expandable introducer 1200 into the human vasculature.

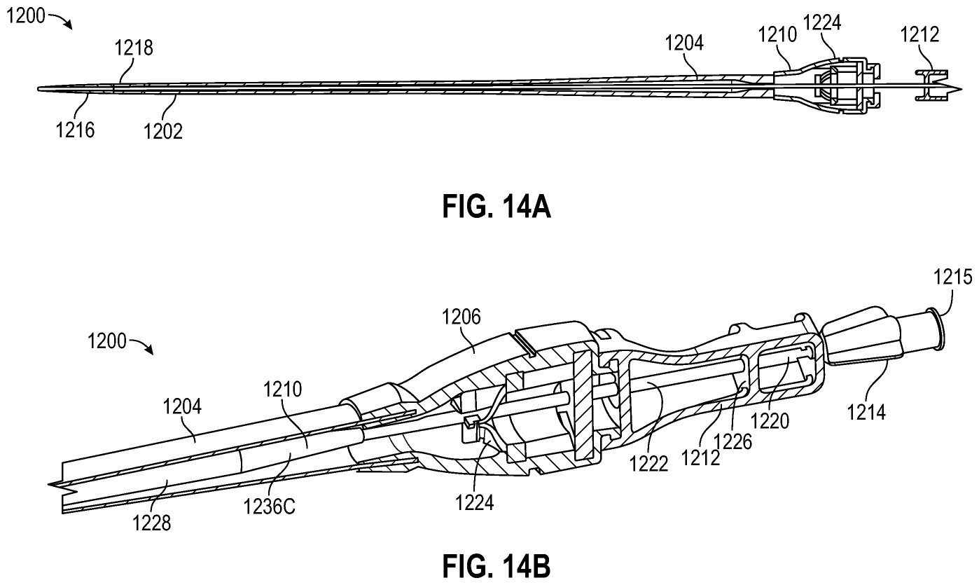

With reference again to FIGS. 12A-12B, the expandable introducer 1200 further comprises an introducer hub 1206 that includes a hemostasis valve 1224 (visible in FIGS. 14A-14B). The introducer hub 1206 is connected to a flushing three-way stopcock 1208. An interior volume of the introducer hub 1206 inside the seal of the hemostasis valve 1224 and including the mesh sheath 1202 (including the first porous region 1203 and the second non-porous region 1204) may be flushed using the three-way stopcock 1208.

With reference to FIG. 12C, the expandable introducer 1200 further comprises a sheath dilator 1210 for obturating the mesh sheath 1202 or providing a removable dilating tip for insertion into the body. The sheath dilator 1210 can be locked in place within the mesh sheath 1202 of the expandable introducer 1200 by a manually removable clip 1212 (visible in FIG. 12B). The clip 1212 engages with the introducer hub 1206 and can be removed by an operator to allow the sheath dilator 1210 to advance or retract through the expandable introducer 1200 in use. At a proximal end of the sheath dilator 1210, a dilator Luer fitting 1214 is provided. The dilator Luer fitting 1214 can be manipulated by an operator to advance or retract the sheath dilator 1210 once the clip 1212 is released, as well as allowing releasable fluid coupling with additional tubing, syringes, pumps, etc. At an opposite distal end of the sheath dilator 1210, a sheath dilator tip 1216 is provided. The tip 1216 is soft and tapered to facilitate entry into the body and also to provide an atraumatic tip during distal advancement through the vessel.

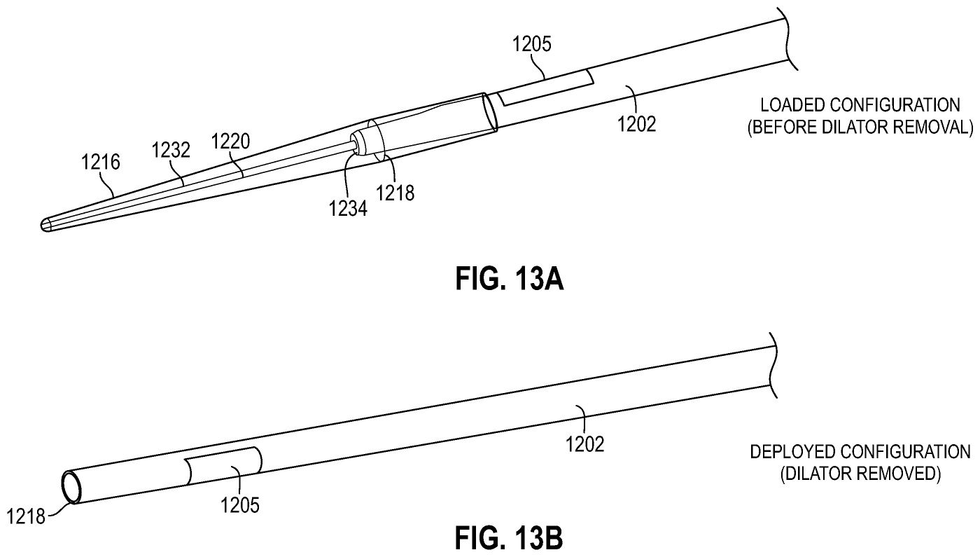

In a loaded configuration of the sheath dilator 1210, seen more clearly in FIG. 13A, the sheath dilator tip 1216 extends past and sits as a cap over a thus-constrained open end 1218 of the mesh sheath 1202. As described more fully below, the expandable introducer 1200 may be advanced into the human vasculature in the loaded configuration and then deployed once in position. In a deployed configuration of the expandable introducer 1200, seen more clearly in FIG. 13B, the sheath dilator 1210 has been advanced through the mesh sheath 1202 to expand the sheath 1202 and push the sheath dilator tip 1216 off the constrained open end 1218 of the mesh sheath 1202. This release of the sheath dilator tip 1216 allows the now-uncapped open end 1218 of the mesh sheath 1202 to enlarge in size. In some examples, the enlargement occurs or is driven by virtue of a natural or inherent resiliency in the material of the mesh sheath. The enlarged open end 1218 of the mesh sheath 1202 is larger than the outer diameter of the sheath dilator tip 1216. As a result, the sheath dilator 1210 is free to be retracted by an operator to withdraw the sheath dilator tip 1216 back through the enlarged open end 1218 of the mesh sheath 1202 and extract the sheath dilator 1210 and the sheath dilator tip 1216 from the expandable introducer 400 as a whole, leaving the sheath open and expanded. The introducer hub 1206 of the introducer 1200 is now ready to accept introduction of the loading tool 502 and the embolic protection device.

In some examples, the introduction of the loading tool is enabled, or at least facilitated, by an introducer hub adapter. An example hub adapter may also be configured to accommodate and provide sealing passage of two (or more) parallelly extending catheters, dilators, or other instruments that are not coaxial and instead reside side by side in use. It will be seen, for example in FIG. 5, that the embolic protection device 300 and the filter dilator 402 are not coaxial and may extend, at least for a portion of their lengths, substantially in parallel.

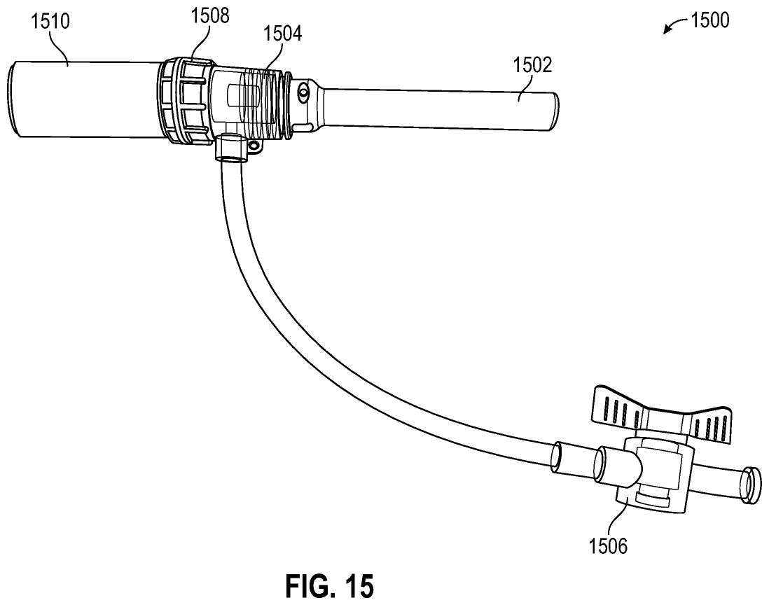

A pictorial view of an example introducer hub adapter 1500 is shown in FIG. 15. The hub adapter 1500 includes an adapter shaft 1502 (or extension tube) that is insertable into the introducer hub 1206 of an introducer 1200. The adapter shaft 1502 may be interchangeable, by means of the screw threads 1504 for example, with another adapter shaft 1502 of a different size to suit the sizes and configurations of various other types of introducer 1200 and/or introducer hub 1206. The adapter shaft 1502 includes a lumen in fluid communication with the interior volume of the introducer hub adapter 1500. A flushing stopcock 1506 is provided to de-air the interior volume of the introducer hub adapter 1500.

In some examples, the introducer hub adapter 1500 includes a multi-catheter seal 1508 located adjacent to an adapter connector 1510. The adapter connector 1510 is configured to accept entry of the tip of the loading tool 502. In some examples, the adapter connector 1510 is lockable to the loading tool 502 by means of a twisting action imposed on the adapter connector 1510. The multi-catheter seal 1508 allows passage of one or more catheters therethrough, but may include an internal inflatable bladder (not shown) that can be inflated by means of an external inflation valve, for example. Inflation of the inflatable bladder causes the walls of the inflatable bladder to seal against the outer surfaces of the one or more catheters passing therethrough. The one or more catheters passing therethrough may include the catheter shaft 302 of the embolic protection device 300, the filter dilator 402, and/or one or more catheters of devices and instruments deployed at the site of an operation in a diseased vessel.

In some examples, other types of multi-catheter seal 1508 may be provided. For example, the multi-catheter seal 1508 may include a mechanically compressible or deformable material, such as a low durometer material, for example a silicon material, which can be deformed to move into and be held in sealing engagement with the outer surfaces of one or more catheters passing through the multi-catheter seal 1508 of the introducer hub adapter 1500. The deformable or compressible material may be compressed by a manually operable restriction band or another device. The mechanically compressible or deformable material may include one or more apertures through which one or more respective catheters may pass. Other arrangements and configurations of a multi-catheter seal 1508 are possible.

With reference to FIGS. 16A-16M, example aspects of preparation and deployment of an embolic protection device 300 and other embolic system components for use in a medical procedure are now described.

In FIG. 16A, an introducer 1200 is loaded onto a guide wire 406, and deployed as described further above. The introducer 1200 may have a fixed inner diameter or be expandable.

In FIG. 16B, an introducer hub adapter 1500 is loaded on to the guide wire 406 and prepared for insertion into the introducer hub 1206 of the introducer 1200.

In FIG. 16C, the embolic protection device 300 is prepared for deployment, with the guide wire 406 passing through the side port 308 of the filter 304.

In FIG. 16D, the filter 304 of the embolic protection device 300 is in an open configuration and the side port 308, including a seal 330, is visible.

In FIG. 16E, the filter dilator 402 is inserted into the filter 304 through the side port 308.

In FIG. 16F, the filter dilator 402 is advanced through the side port 308 such that the circumferential channel 404 in the tip 412 of the filter dilator 402 lies substantially adjacent the hoop wire 318.

In FIG. 16G, the hoop wire 318 is tightened by manipulation of the actuation slider 314 in the filter actuator 306 such that the mouth 322 of the filter 304 closes in on and is entrapped in the circumferential channel 404, and the actuation slider 314 is locked in the closed position.

In FIG. 16H, the filter 304 is loaded into the loading tool 502.

In FIG. 16I, the loading tool 502 is locked (the hub 506 is engaged) and prepared for insertion into an introducer (not visible in the view) with or without an introducer hub adapter.

In FIG. 16J, an introducer hub adapter 1500 is used, and the tip of the loading tool 502 is inserted into the introducer hub adapter 1500. An enlarged view of this arrangement is provided in FIG. 16K. The introducer hub 1206 of the introducer 1200 is visible in both views.

In FIG. 16L, the filter 304 of the embolic protection device 300 is pushed through the introducer 1200 and the loading tool is removed (peeled away).

In FIG. 16M, a therapeutic device (such as a TAVR device 1600) is tracked over the guide wire 406 through the side port 308 of the filter 304 and out through the open mouth 322 of the filter 304 and moved to a target treatment region.

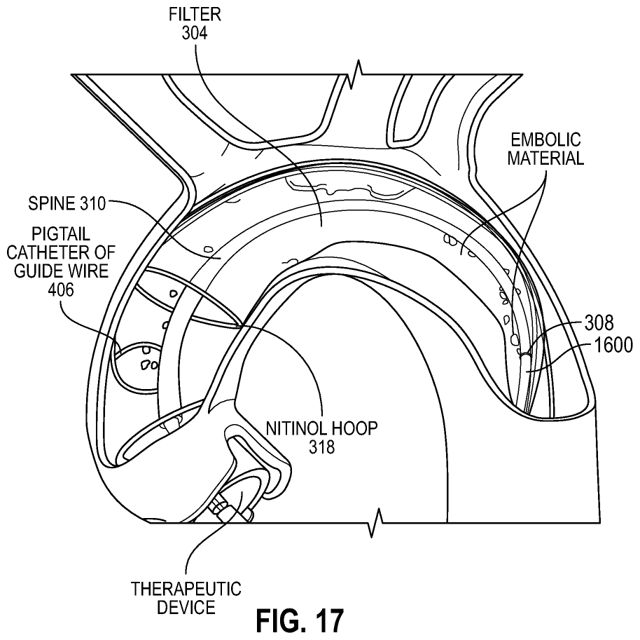

FIG. 17 shows a pictorial view of a therapeutic device deployed by an embolic protection device 300 and other components of an embolic protection system at the target treatment region. An example TAVR 1600 device is shown passing through the side port 308.

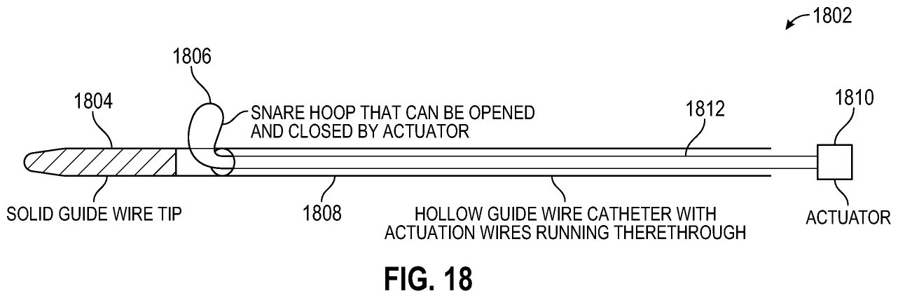

FIG. 18 shows a schematic view of a snare guide wire 1802 that can be used in conjunction with an embolic protection device and/or other embolic system components and TAVR devices in a medical procedure, according to example embodiments.

The snare guide wire 1802 includes, at a leading or distal end thereof, a solid guide wire tip 1804. The snare guide wire 1802 also includes a snare hoop 1806 that can open and close under action of an actuator 1810. The actuator 1810 controls the snare hoop 1806 by means of actuation wires 1812 passing through a hollow guidewire catheter 1808 of the snare guide wire 1802. The snare hoop 1806 can be manipulated (opened or closed) by an operator using the actuator 1810 to capture or engage other guide wires (such as a regular guide wire) and other components (such as a TAVR device) to assist in passing these other guide wires and components through a side port (or side window) of an expandable filter of an embolic protection device, for example an embolic protection device of the one or more examples described above.

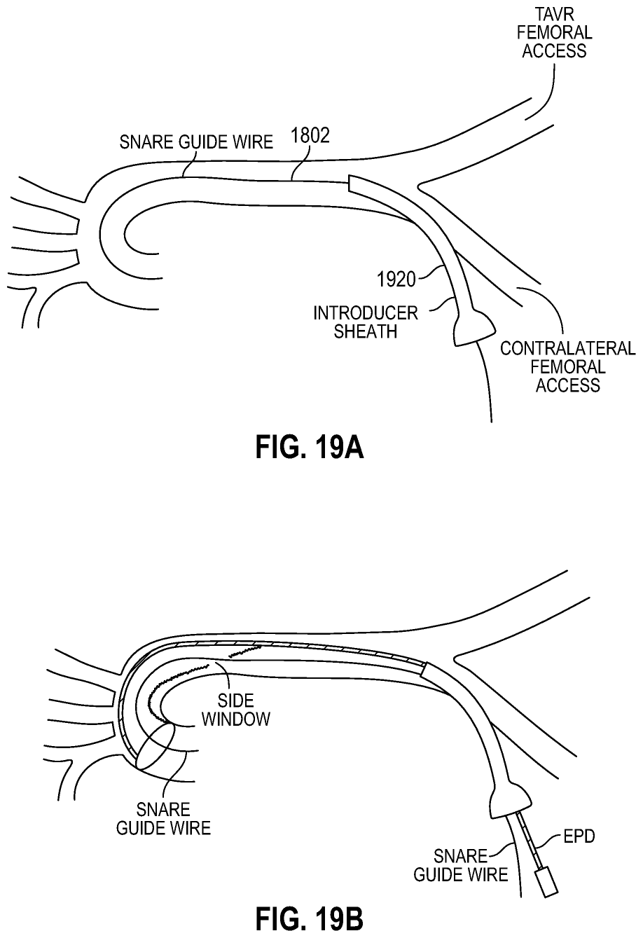

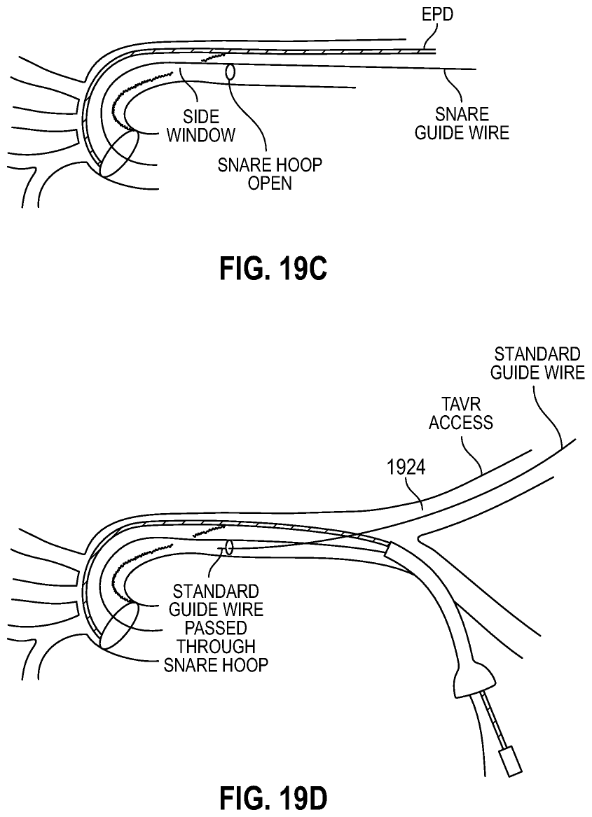

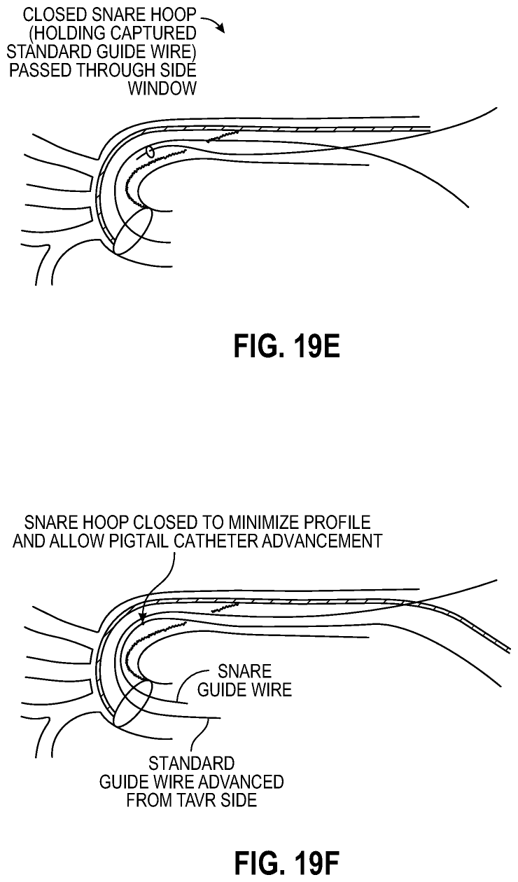

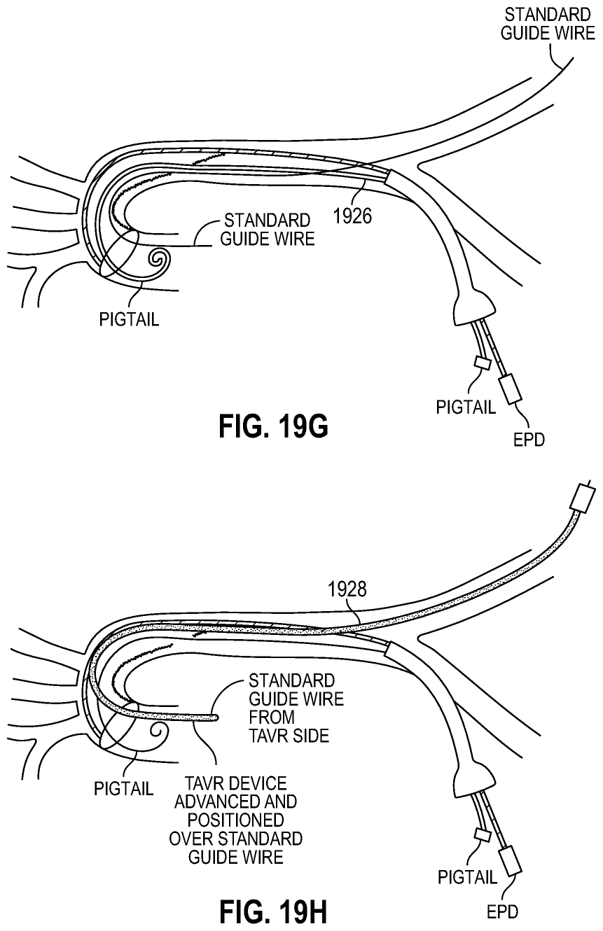

Some of these facilitation methods involve establishing access to the human vasculature via a femoral artery (for example at a TAVR femoral access point, FIG. 19A) and a contralateral femoral artery (for example at a contralateral femoral access point, FIG. 19A). An example procedure is described below.

FIGS. 19A-19H show various aspects and operations in the preparation and deployment of a snare guide wire 1802 used in conjunction with an embolic protection device and other embolic system components, guide wires and therapeutic devices (such as a TAVR device) in a medical procedure, according to example embodiments.

In FIG. 19A, the snare guide wire 1802 is inserted into the human vasculature through an introducer sheath 1920 via contralateral femoral access. The introducer sheath 1920 may include a mesh sheath of an introducer 1200 described further above.

In FIG. 19B, an embolic protection device (such as an embolic protection device 300 described further above) is inserted over the snare guide wire 1802 via contralateral femoral access.

In FIG. 19C, the snare hoop 1806 of the snare guide wire 1802 is aligned just outside the side window (for example a side port 308 described above) of the filter of the embolic protection device (for example a filter 304 as described above) and the snare hoop 1806 is opened.

In FIG. 19D, a standard guide wire 1924 is advanced from the TAVR femoral access so the tip of the standard guide wire 1924 is in the open snare hoop 1806.

In FIG. 19E, the snare hoop 1806 is closed to trap the tip of the standard guide wire 1924, and an operator advances the snare guide wire 1802 through the filter side window which also brings the standard guide wire 1924 through the side window. Passage of the standard guide wire 1924 though the side window is thus facilitated by the snare guide wire 1802. Other components may be passed though the side window in similar manner, in some examples.

In FIG. 19F, the snare hoop 1806 is opened to release the standard guide wire 1924. The released standard guide wire 1924 can then be advanced to a desired location through the filter, as shown. The snare hoop 1806 is then closed to minimize its profile to ease or allow passage of other components and guide wires, for example as described below.

In FIG. 19G, a pigtail catheter 1926 is advanced over the snare guide wire 1802 (with the snare hoop 1806 closed) and the snare guide wire 1802 is then removed.

In FIG. 19H, a therapeutic device, for example a TAVR device 1928, can then be advanced over the standard guide wire 1924 from the TAVR femoral access, through the filter.

In summary, as described above, the inventive protective configurations of the embolic protection device and system provide a means for conducting an intervention while also protecting the underlying tissue and related anatomy.

Various exemplary embodiments of the invention are described herein. Reference is made to these examples in a non-limiting sense. They are provided to illustrate more broadly applicable aspects of the invention. Various changes may be made to the invention described without departing from the scope of the invention as defined by the appended claims. In addition, many modifications may be made to adapt a particular situation, material, composition of matter, process, process act(s) or step(s) to the objective(s), or scope of the present subject matter. Further, as will be appreciated by those with skill in the art that each of the individual variations described and illustrated herein has discrete components and features which may be readily separated from or combined with the features of any of the other several embodiments without departing from the scope of the present embodiments. All such modifications are intended to be within the scope of claims that may be associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The present application discloses methods that may be performed using the subject devices. The methods may comprise the act of providing such a suitable device. Such provision may be performed by the end user. In other words, the “providing” act merely requires the end user obtain, access, approach, position, set-up, activate, power-up, or otherwise act to provide the requisite device in the subject method. Methods recited herein may be carried out in any order of the recited events that is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the present disclosure in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described without departing from the scope of the invention defined by the appended claims. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only,” and the like in connection with the recitation of claim elements, or use of a “negative” limitation.

Without the use of such exclusive terminology, the term “comprising” in claims associated with this disclosure shall allow for the inclusion of any additional element, irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure as defined by the appended claims.

1. An embolic protection system for deploying a device in a diseased vessel of a vasculature, the embolic protection system comprising:
an embolic protection device including:
a catheter shaft;
an expandable filter provided at a distal end of the catheter shaft, the expandable filter including a side port provided in a side wall of the expandable filter; and
a filter actuator located at or towards a proximal end of the embolic protection device, the filter actuator operable to open and close a mouth of the expandable filter.

2. The embolic protection system of claim 1, further comprising:
a dilator.

3. The embolic protection system of claim 1, further comprising:
a loading tool.

4. The embolic protection system of claim 1, further comprising:
an introducer.

5. The embolic protection system of claim 4, further comprising:
an introducer hub adapter.

6. The embolic protection system of claim 4, wherein:
the introducer is an expandable introducer

7. A method of deploying an embolic protection device into a vasculature of a patient, the method comprising:
establishing access to the vasculature;
introducing a guide wire into the vasculature to assist with guidance of the embolic protection device through the vasculature;
advancing the embolic protection device into the vasculature and over the guide wire toward a target treatment region, wherein the embolic protection device comprises a catheter shaft, an expandable filter disposed at a distal end of the catheter shaft wherein the expandable filter includes a side port provided in a side wall of the expandable filter, and a filter actuator disposed adjacent a proximal end of the catheter shaft, wherein the filter actuator is operable to open and close a mouth of the expandable filter;
actuating the filter actuator thereby radially expanding the expandable filter at the target treatment region and opening the mouth of the expandable filter; and
capturing emboli in the expandable filter.

8. The method of claim 7, further comprising inserting a therapeutic device through the embolic protection device and advancing the therapeutic device toward the target treatment region.

9. The method of claim 7, wherein the target treatment region comprises a native aortic valve.

10. The method of claim 8, further comprising, after inserting the therapeutic device through the embolic protection device, closing the expandable filter and withdrawing the embolic protection device from the vasculature.

11. The method of claim 1, further comprising introducing a snare guide wire into the vasculature to assist with guidance of a guide wire or a device through the side port of the embolic protection device.
